(19) United States
(12) Patent Application Publication
(10) Pub. No.: US 2012/0165837 A1
(43) Pub. Date: Jun. 28, 2012

(54) Endoscopic Suturing Device, System and Method

(75) Inventors: Yuri Belman, Campbell, CA (US); Alexander Borisovich Zatyuryukin, Moscow (RU); Patricia A. Moore, Incline Village, NV (US)

(73) Assignee: Suturenetics, Inc., Incline Village, NV (US)

(21) Appl. No.: 13/168,426

(22) Filed: Jun. 24, 2011

Related U.S. Application Data
(60) Provisional application No. 61/358,764, filed on Jun. 25, 2010.

(51) Int. Cl. A61B 17/04

(52) U.S. Cl. 606/144

(57) ABSTRACT

Improved medical suturing devices, systems, and methods may hold a suture needle at a fixed location relative to a handle of the device, allowing a surgeon to grasp and manipulate the handle of the suturing device to insert the needle through tissues. The exemplary device includes two needle grasping clamps extending from an elongate distal portion for endoscopic surgeries, including ear, nose and throat procedures. The two clamps alternate holding the suture needle, as the surgeon sutures the tissues, each clamp having a proximal and distal gripping jaw for grasping the needle. Preferably, the gripping surfaces of the proximal and distal gripping jaws are substantially parallel to the needle's plane of curvature and exert a holding force on the needle substantially along an axis of the device.

### CROSS-REFERENCES TO RELATED APPLICATIONS

The present application claims the benefit under 35 USC 119(e) of U.S. Provisional Application No. 61/358,764 filed Jun. 25, 2010; the full disclosure of which is incorporated herein by reference in its entirety for all purposes.

The following commonly-assigned applications and patents disclose related subject matter, and are hereby incorporated herein by reference: U.S. patent application Ser. No. 11/532,032 filed Sep. 14, 2006 and titled “Suturing Device, System, and Method;” U.S. patent Provisional application Ser. No. 11/227,981 filed Sep. 14, 2005; U.S. patent application Ser. No. 12/535,499 filed Aug. 4, 2009; U.S. patent application Ser. No. 12/049,552 filed on Mar. 17, 2008; and U.S. patent application Ser. No. 12/049,545 filed on Mar. 17, 2009.

### BACKGROUND OF THE INVENTION

The present invention generally relates to medical devices, systems, and methods. In specific embodiments, the invention provides devices, systems, and methods for suturing tissues in open surgery, minimally invasive surgical procedures, robotic surgery, and the like.

Although many aspects of surgery have changed radically over the last several decades, some surgical techniques have remained remarkably constant. For example, as was true fifty years ago, suturing remains a common technique for approximation of tissues, ligation of tissues, affixing tissues together, and the like.

Suture has been used in open surgical procedures for generations to therapeutically treat diseased tissue and to close surgical access sites and other wounds. More recently, the use of minimally invasive surgical techniques has expanded, with surgical therapies often being performed at internal surgical sites. Although a wide variety of visualization techniques (including laparoscopes and other endoscopic viewing devices, fluoroscopy and other remote imaging modalities, and the like) have been developed to allow surgeons to view these internal surgical sites, and although a large variety of new tissue treatment techniques have been developed (including ultrasound techniques, electrosurgical techniques, cryosurgical techniques, and the like) and are now widely available, many modern surgical interventions continue to rely on suturing.

A wide variety of alternatives to suturing of tissues have been developed, and have gained varying degrees of acceptance in certain surgical procedures. Staples and tissue adhesives are used quite frequently in many open and minimally invasive surgical settings, and a variety of tissue welding techniques have also been proposed. Nonetheless, suturing remains ubiquitous in surgery, as suturing provides a number of advantages over many of the alternatives.

Suture's advantages include the large knowledge and skill base that surgeons have developed over the years. Additionally, a variety of off-the-shelf, pre-packaged surgical needles with suture are available from a large number of suppliers at very reasonable cost. Surgeons are able to precisely control the location of suture stitches by grasping the suture needle and first pushing it and then pulling it through the target tissue. In open surgery the surgeon may manually grasp the suture needle directly with his or her hand, although both open and minimally invasive procedures are often performed by grasping the needle with a needle grasping tool and manipulating the tool to place the suture stitches. The results obtained using suture are highly predictable, although dependent on the skill of the surgeon. In light of its advantages, the use of suture does not appear likely to disappear any time soon, with even modern robotic surgical techniques often making use of suture.

Although suture remains popular in surgery at least in part due to its significant advantages, suturing is not without disadvantages. In particular, placing a large number of suture stitches can be tiring and quite time-consuming. Manipulation of a suture needle can be difficult even in open surgery due to the limited space that is often available around the target tissues. The challenges of manipulating suture needles may be even greater in minimally invasive surgical procedures, where the needles are often manipulated using long-handled tools extending through a small aperture, typically while viewing the procedure on a display which is offset from the surgical site. Tying knots with a desired amount of tension and the like may call for intricate and precise manipulation of the suture, further complicating and delaying open and minimally-invasive surgeries. In fact, the time spent closing/suturing the access site may be significantly greater than the time spent treating the underlying target tissues for many procedures. The challenges of suturing tissue may be particularly acute during minimally invasive procedures, as such procedures often seek to limit trauma by relying on a limited number of relatively small access apertures to an internal surgical site. While endoscopic and other minimally invasive surgical techniques can provide tremendous advantages to the patient, these advantages may rely on increases in both the skills of the surgeon and the time spent suturing using known endoscopic needle graspers and the like.

There have been a variety of proposals for modifications to standard surgical suturing structures and methods to try to address the above disadvantages. At least some of these proposals may seek to rely on specialized and/or proprietary suturing needle systems, which could increase costs and preclude their wide acceptance, especially in third world countries. Unfortunately, many proposals for modifying existing suturing techniques may also decrease the surgeon's control over the placement of the suture, such as by relying on an automated or indirect mechanical movement of a device to drive a suture needle into and/or through tissues. While these new proposals have in the past or may in the future gain varying degrees of acceptance in one or more surgical procedures, standard suturing techniques continue to predominate throughout surgery in general.

In light of the above, it would be desirable to provide improved suturing devices, systems, and methods. It would be generally desirable to maintain some, most, or all of the advantages of standard suturing techniques, preferably while decreasing the time required for suturing, the strain on the surgeon, the training involved in achieving competence or time-efficiency in suturing techniques, or the like. It would be particularly advantageous if these improvements could be provided for minimally invasive and/or open surgical procedures, optionally without requiring extensive capital investments for new equipment, without significant increases in complexity of the suturing process, or without having to resort to specialized or proprietary suturing needles and the like. Alternative needle grasper structures which increased the ease and accuracy of stitching, which facilitate and expedite suturing within an internal surgical site through a limited number of cannulae, trocars, or other minimally invasive surgical access tools, and/or which are readily adapted for a variety of different procedures and patient physiologies would also be desirable.

### BRIEF SUMMARY OF THE INVENTION

The present invention generally provides improved medical suturing devices, systems, and methods. Embodiments of the invention provide improved suturing systems, devices and methods that maintain some or all of the advantages of standard open and/or minimally invasive suturing techniques while providing enhanced speed and ease of use. While some embodiments will find uses in a wide range of open surgical procedures, many advantageous embodiments will be particularly useful for minimally or less invasive surgeries, otolaryngology, pediatric surgeries, endoscopic surgeries (with or without trocar access), laparoscopic surgeries, and/or other procedures in which access to a suture site is limited. Articulation motions may be transferred from a handle to a needle grasping clamp using an axial movement of an actuation shaft that is loaded in compression along an elongate axis of the device and within an outer body or sheath, although alternative embodiments may make use of actuation cables loaded in tension. The device includes two alternating clamps that both advance axially and rotate to grasp the needle, the gripping forces on the needle being substantially applied along a longitudinal axis of the device and the grasping surface being substantially parallel to the needle's plane of curvature. Gripping the needle so that the needle is stressed slightly when the clamps alternate can be advantageous as it may inhibit “walking” of the needle in the longitudinal direction that may occur as the clamps alternate holding the needle. In some embodiments, the jaws may expand or unfold laterally after insertion to facilitate passing the clamps of the device axially through a trocar in preparation for an endoscopic procedure. This approach allows for use of clamps having relatively large jaw openings that can more easily accommodate larger needles and larger bite distances desirable for suturing thicker tissues.

In a first aspect, the invention provides a suturing device for use with a suturing needle. The device comprises a body having a proximal portion and a distal portion. The distal portion comprises the working portion having clamps that suture the tissues, the distal direction being toward the tissues of the patient. The proximal portion is held by the surgeon to control the device, the proximal direction being toward the surgeon. The body includes a first and second shaft that extend from the distal portion and are movable axially along the device axis and rotatably about each shaft. A first and second clamp are supported by the first and second shafts, respectively, and may be mounted at a distal end of the shaft, the shaft extending distally from the body. Each clamp comprises a proximal and distal jaw, the distal jaw being distal of the proximal jaw along an axis of the device. Movement of the first and second clamps is effected by a linkage within the body of the device. When actuated, the linkage causes movement of the clamps between a retracted displaced position and a grasping position by axially moving and rotating each shaft. When in the grasping position, each clamp grasps a portion of the needle disposed between the proximal jaw and the distal jaw along the device axis. When in the retracted position, each clamp is rotated laterally away from the needle and refracted proximally to increase clearance between the retracted clamp and the tissue and/or needle. In some embodiments, any or all of the clamps, jaws and shafts may be made of a rigid material.

Optionally, the clamps, shafts, and linkage effecting movement of each of the clamps comprise a detachable cartridge. The detachable cartridge facilitates sterilization or customization of the device with different cartridges. Cartridges may differ in various ways, including the dimensions of the shafts and/or clamps and the configurations of the clamps. In some embodiments, the linkage effecting movement of each of the first and second clamps includes a compression rod. The linkage may also include one or more springs to effect movement of the first or second clamps, providing a spring force for opening or closing of the jaws of the clamp or moving the movable handle.

In a preferred embodiment, the suturing device includes a handle at the proximal end of the body. The handle is coupled to the first and second clamps by the linkage mechanism so that an actuation of the handle alternates between: the first clamp in the grasping position while the second clamp is in the retracted position; and the second clamp in the grasping position while the first clamp is in the retracted position. Preferably in grasping the needle, the linkage axially advances the first or second clamp along an elongate axis of the device, rotates the clamp about an axis of a shaft supporting the clamp (with the clamp being offset from the shaft axis so that the clamp moves laterally toward an axis of the needle), slightly advances the clamp axially along the axis of the body of the device so as to stress the needle, and then closes the clamp on a portion of the needle. Ideally, an actuation of the handle comprises the handle moving from a first position to a second position, typically performed by a physician squeezing the handle with one hand. Often, for a short time after one clamp grasps the needle during the actuation cycle, both clamps are holding a portion of the needle.

Optionally, each clamp opens after the other clamp has closed during an actuation cycle so that the clamps maintain the needle at a substantially fixed location relative to the body of the suturing device. The linkage effecting movement causes each of the first and second clamps, at different times during a cycle, to advance toward the needle and rotate laterally toward the needle. Ideally, the clamp rotates toward the body in the same plane as the curvature of the needle so as to grasp the needle between the jaws of the clamp. The jaws of the clamp may include a gripping surface positioned to grip primarily (and often almost entirely) in a direction along the device axis. The jaws may also include a chamfered projection at an end of a jaw element to nudge the needle into alignment with the gripping surfaces of the jaws so long as the needle is within a grasping area of the jaws.

In some embodiments, at least one (and preferably both) of the clamps of the suturing device has a folded configuration and a working configuration. In the folded configuration, the components of the clamp fold so as to reduce the overall profile of the device and facilitate introduction of the device through a trocar. In the working configuration, the clamp unfolds to facilitate normal operation of the clamp during the suturing process as described above. In embodiments having foldable clamps, the suturing device may also include a sheath that at least partially houses the clamps and the body in the folded position. A physician may rotate the sheath to either fold the clamps into a folded position or to unfold the clamps into the working configuration. Typically, after unfolding the clamps, the clamps are locked into the working configuration by a locking mechanism, which can be released to facilitate folding the clamps by pressing a release button. The sheath may also include tabs which direct the clamps between the configurations as the sheath is moved or rotated.

In yet another aspect, the invention provides a method for suturing. The method comprises introducing a body of a suturing device to a surgical site for a patient. The body extends along a device axis toward first and second clamps disposed near its distal end. A base portion of a surgical needle is grasped between a distal jaw element and a proximal jaw element of the first clamp along the device axis. The sharp end of the needle is inserted through a tissue of the patient while the first clamp holds the needle at a fixed location relative to the body. The second clamp is then advanced toward the needle by translating a shaft supporting the second clamp along the device axis and rotating the second clamp about its shaft. The second clamp then grasps a tissue penetrating portion of the needle, and the first clamp releases the base portion of the needle withdrawing into a retracted position by rotating about its shaft and translating along the device axis. The base end of the needle is then pulled through the tissue of the body by moving the body of the suturing device while the second clamp holds the needle.

Often, inserting the sharp end of the needle through the tissue and pulling the base end of the needle through the tissue comprises a physician or surgeon manually holding a proximal portion of the suturing device, such as a handle, and moving or rotating the proximal portion of the device. Typically, grasping a needle with a clamp includes advancing the clamp axially along the device axis toward the needle to a position lateral of the needle, rotating the clamp laterally toward the needle such that the clamp partially surrounds a portion of the needle, and then closing the clamp on the needle. Allowing the physician to drive the needle by manually moving or rotating a proximal portion of the suturing device enables the physician to retain more control over suturing of the tissue. This is advantageous as the physician may need to alter the suturing process in various circumstances. For instance, if the physician notices bleeding when the needle is first inserted into a tissue, indicating the needle was inserted into a vessel, the physician can simply stop driving the needle and remove the needle by pulling the partially inserted needle back out along the insertion path using the clamp engaging the base portion to prevent further damage to the vessel. Additionally, this aspect of the device allows the physician to retain the “feel” of the needle as it drives through the tissue, which may help the surgeon locate or avoid various tissues. A method of suturing with the suturing device may include selecting an insertion point in the tissue to be sutured, inserting the needle in the tissue at the insertion point, selecting a second insertion point in the tissue, inserting the needle into the tissue at the second insertion point, and selecting and inserting the needle into the tissue at additional insertion points until the tissue suture is complete. The method may include removing the needle from any insertion point by pulling the partially inserted needle back out along the insertion path if the physician desires to select an alternate insertion point in the tissue. For instance, a physician may wish to remove the needle and choose an alternate insertion point in the tissue if the needle enters a vessel causing bleeding or the needle enters a tissue or region, which the physician would prefer to avoid suturing.

In many embodiments, the suturing method includes axially stressing the needle along its axis with the clamp before closing the clamp on the needle. Often, the proximal jaw of the clamp is used to axially stress the needle by slightly advancing the proximal jaw toward the needle before the clamp closes on the needle. Ideally, grasping a portion of the needle with the clamp comprises grasping the needle from within the plane of the needle's radius of curvature. In another aspect of the invention, the jaws of the clamp exert a gripping force on the needle substantially in the direction of the device axis. The method may also include simultaneously holding the needle with both clamps before releasing the needle with either clamp, which may prevent inadvertently releasing the needle in a body cavity of the patient. Preferably, when the needle is held by both clamps at once, the gripping force exerted by one clamp on the needle may increase while the gripping force exerted by the other clamp decreases, the increase being roughly proportional to the decrease in gripping force.

In an alternative exemplary embodiment, the method may further include moving the clamps from a folded configuration to a working configuration and vice versa. The clamps may be folded and unfolded by rotating a sheath about the clamps, wherein the sheath may have tabs to guide or facilitate movement of the clamps from one configuration to another. After the clamps are unfolded, the clamps may be locked into the working configuration with a locking mechanism, which may later be released before folding the clamps.

### BRIEF DESCRIPTION OF THE DRAWINGS

FIG. 1 shows an exemplary embodiment of a suturing device.

FIG. 2 shows the suturing device with a detachable distal portion detached from a proximal handle portion.

FIG. 3 shows two clamps of the suturing device, one clamp in an extreme distal position grasping a needle and the other clamp retracted into an extreme proximal position.

FIG. 4 shows an exemplary clamp having two jaws for grasping the needle.

FIG. 5 shows an exploded view of a clamp.

FIG. 6 shows an exemplary clamp, and illustrates how the proximal movement of a push rod opens the jaws of the clamp so as to release a needle.

FIG. 7 shows an exemplary clamp, the distal movement of a push bar causing the jaws of a clamp to close and grasp a needle.

FIG. 8 shows an exemplary clamp, the proximal movement of a push bar causing the jaws of the clamp to open.

FIG. 9 shows a suturing device with both clamps in the extreme proximal position.

FIG. 10 shows the suturing device with one clamp in an extreme distal position grasping a needle and the other clamp refracted in an extreme proximal position.

FIG. 11 shows the suturing device with one clamp grasping the base of the needle, the rotational movement of the device driving the sharp end of the needle through the tissue.

FIG. 12 shows the suturing device with one clamp grasping the needle near the sharp end.

FIG. 13 shows individual components of the linkage within the distal portion of the suturing device that effect movement of the clamps.

FIG. 14 shows an exploded view of the individual components of the linkage within the distal portion of the suturing device.

FIG. 15 shows a push bar and helical bushing that imparts rotational movement to the shaft of the clamps when the shaft is advanced.

FIG. 16 shows a push bar and stop plate of the linkage in the distal portion of the suturing device.

FIG. 17 shows individual components of the linkage within the proximal portion of the suturing device that effect movement of the clamps.

FIG. 18 shows an exploded view of the individual components of the linkage within the proximal portion of the suturing device.

FIG. 19 shows individual components of the linkage that cause axial movement of linkage components to alternate between the shafts of the clamps.

FIGS. 20-22 show an alternative embodiment of the clamps of the suturing device.

FIGS. 23-26 show an alternative embodiment of the clamps of the suturing device.

FIGS. 27-29 show an alternative embodiment of the clamps, each clamp having a folded and unfolded configuration.

### DETAILED DESCRIPTION OF THE INVENTION

The present invention provides improved medical devices, systems, and methods for the application of surgical sutures. Properly realized, the invention facilitates endoscopic and/or open techniques for suturing tissues, which can significantly increase the speed and enhance the simplicity of suture application, especially in cases when the suturing of a long incision is desired.

This invention should find extensive use in tissue suturing during surgical operations on both humans and animals. In addition to endoscopic procedures (for example, during laparoscopy), the subject invention can be used during operations that involve limited access and in other surgical areas where tissue joining is desired. It provides particular advantages in the suturing of a large incision by increasing the speed and improving the ease with which stitches are completed, and with which knots are tied. The devices and related techniques described here can be used, for example, to suture different layers of anatomical tissues, including (but not limited to) various organs (among them, the intestines and the uterus), and so forth. A wide range of blood vessels, including veins and arteries, can also be connected using the techniques described in this document in order to form anastamoses and so on. In addition to increasing the speed and/or facilitating the creation of a surgical suture, devices based on the subject invention enhance a physician's control over the suture application process by maintaining a fixed relationship between the movement of the surgeon's hand and the surgical needle. A version of the invention can be used in automated systems, for example, as an actuator in a robotic system.

The devices described here provide for standard sterilization techniques used for surgical instruments, which implies reuse. Sterilization can be accomplished using an autoclave, as well as chemical sterilization techniques, irradiation, etc., since most or all of the device parts can optionally be made from materials suitable for repeated sterilization (such as stainless steel, other metals, alloys, etc.). Alternatively, a detachable portion of (or an entire) a device for applying sutures may include one or more parts made from a plastic suitable for surgical instruments, with such a portion or device optionally being disposable.

Suturing devices based on the subject invention make it possible to employ standard suture materials with the needles normally used in surgery, for example, needles with flat gripping surfaces, as well as needles with a round, triangular, or other cross-sections. Typically, the surgical needle will have a radius of curvature that often includes a base portion and a sharp penetrating portion.

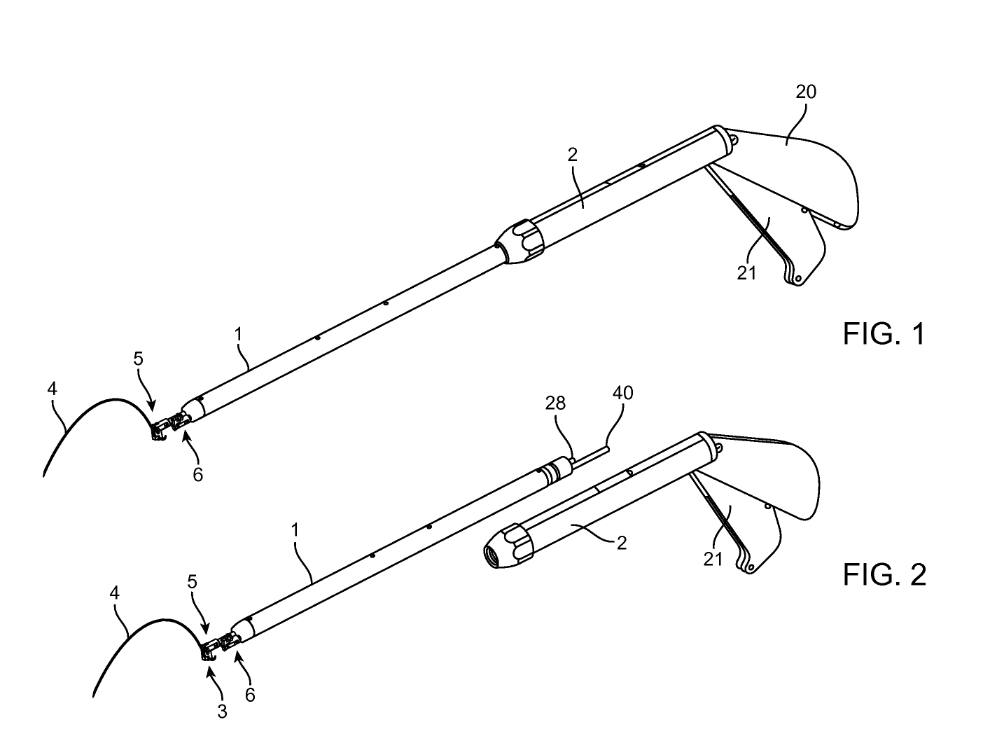

Referring now to FIGS. 1 and 2, an exemplary embodiment of the suturing device includes a body having a distal portion 1 and a proximal portion 2. The distal and proximal portions can be fabricated as an integrated whole or as separate units that can be joined before surgery by a quick-disconnect coupling, as shown in FIG. 2, for example. The detachable distal portion 1 may be disposable or may have several modifications for different applications. The distal portion 1 comprises two compression rods 28, 40 coupled to two clamps 5 and 6. When the assembled device is repeatedly actuated, the clamps may alternate holding a surgical needle 3 so that a physician can suture a tissue with thread 4. The proximal portion 2 comprises a handle 20 by which the physician holds the suturing device and a movable handle 21 by which the surgeon actuates clamps 5 and 6 of the device to suture a tissue, particularly during an endoscopic procedure.

The distal portion 1 of the suturing device body typically comprises a long, narrow body or working part with a round cross-section. The application for a device determines the dimensions of distal portion 1. For instance, the distal portion 1 of a device for endoscopic procedures can consist of a long narrow working part, having a cross-section that can be inserted through a trocar, and a length that ensures suture application at the desired depth.

The proximal portion 2 of the body includes a handle 20 by which a physician can hold the suturing device and a movable handle 21 by which the physician can actuate the suturing device.

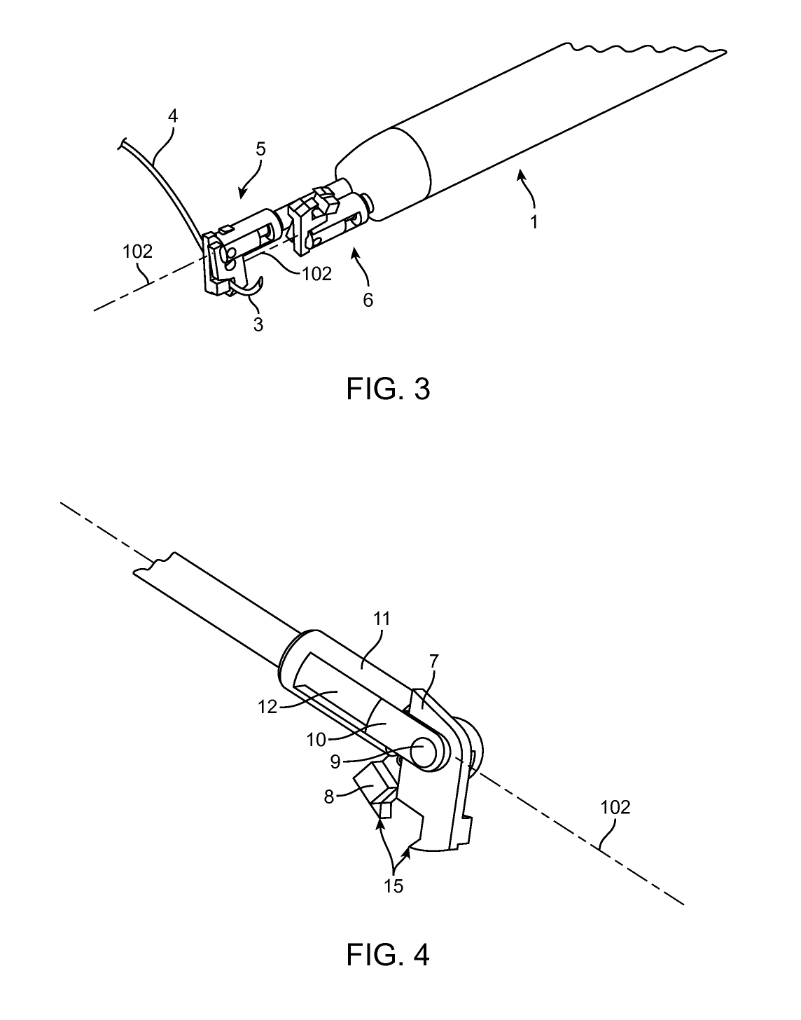

The two clamps of the suturing device may be used with a standard surgical needle 3, usually curved in shape, to the base of which a suturing thread 4 is attached. The needle's dimensions can be selected in accordance with the type of tissues being joined. The two clamps, clamps 5 and 6, shown in FIG. 3 for example, are located at or near the distal end of distal portion 1. In the embodiment of FIG. 3, clamp 5 grips the needle near a base of the needle where thread 4 is attached, while the other clamp 6 sits open in a retracted position. When actuated, clamps 5 and 6 alternate holding the needle. When clamp 6 grips the needle 4 along an insertion portion near the sharp end, clamp 5 retracts to an extreme proximal. Ideally, clamps 5 and 6 are identical, allowing a needle to be clamped for suturing both from right to left, as shown in FIG. 3, and from left to right, if the surgeon held the handle in the left hand, for example.

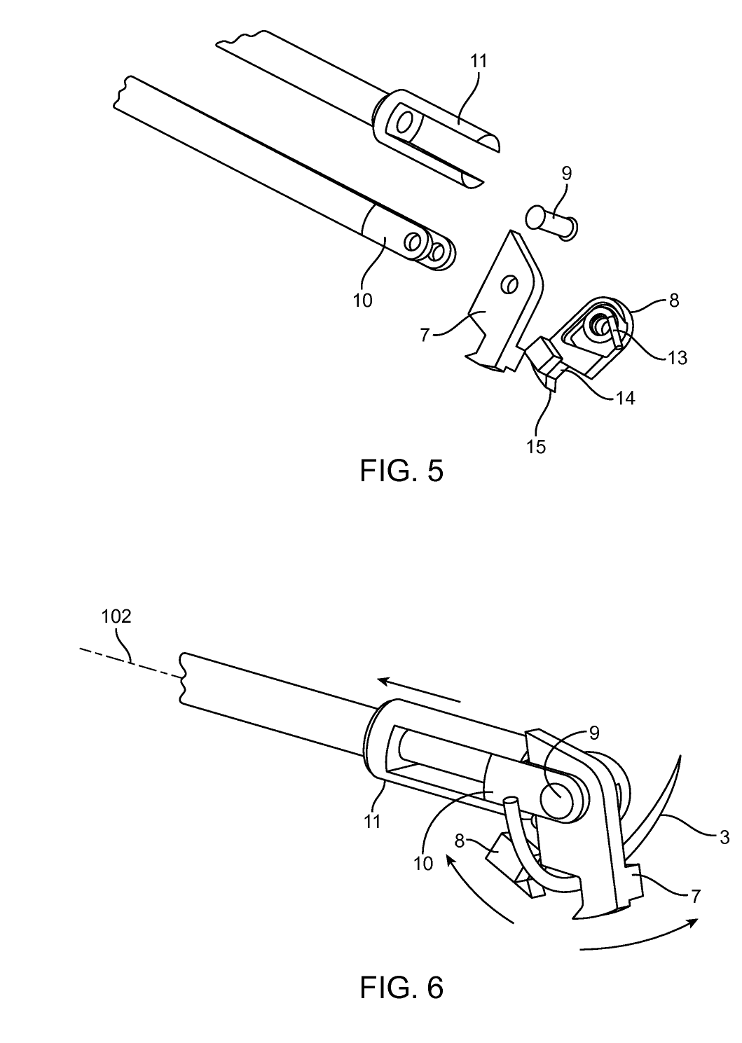

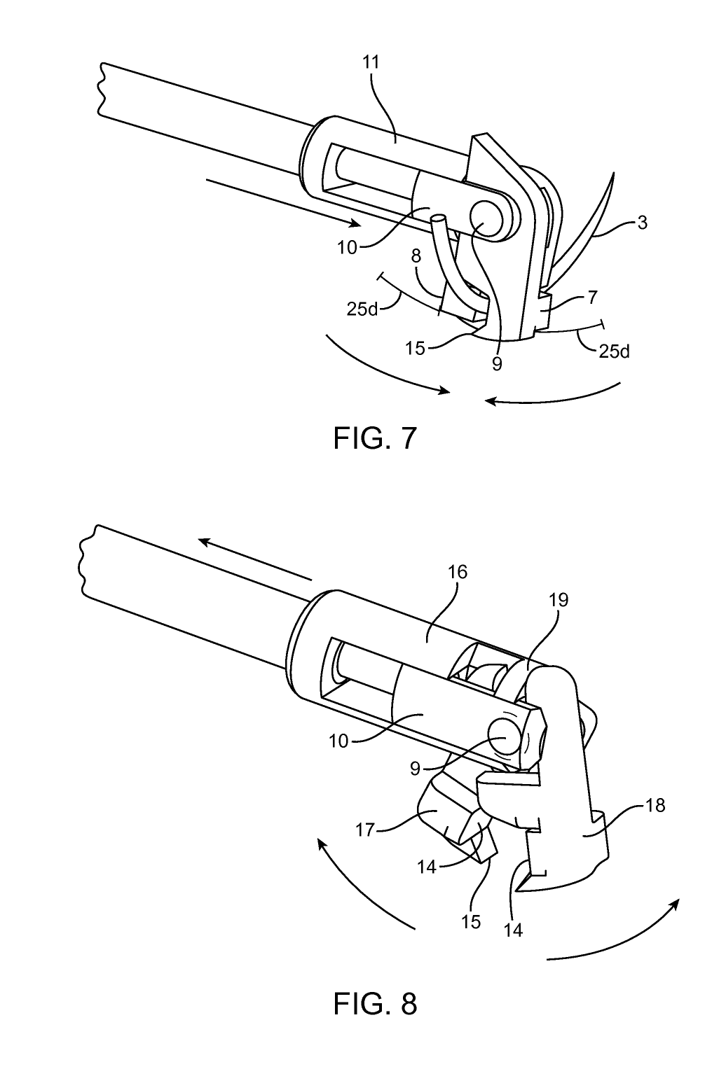

Each clamp comprises two jaw elements, jaws 7 and 8, which are mounted to a pin 9 associated with the clamp and held in place by a yoke 10, which is in turn attached to the end of clamp shaft 12, as depicted in FIGS. 4 and 5 for example. Clamp shaft 12 and yoke 10 are housed inside a tubular push bar 11 that has the ability to move axially. Clamp shaft 12 has a shaft axis 102 around which the clamp can rotate, as shown in FIGS. 3 and 4 for example. In this configuration, a spring 13 is located between jaws 7 and 8 to hold them in the open position. When a compression rod of a clamp is moved in the distal direction, push bar 11 bears on the side surfaces of jaws 7 and 8, making them turn toward one another on pin 9, as illustrated by the directional arrows in FIG. 7. As push bar 11 moves in the proximal direction, jaws 7 and 8 open under the influence of spring 13, illustrated by the directional arrows in FIG. 6 for example. Jaws 7 and 8 grip and hold needle 3, so that the needle is grasped axially between the surfaces 14 of the jaws. Ideally, when a needle is being held, jaw surfaces 14 are positioned substantially parallel to the needle's plane of curvature. In order to ensure that the needle is reliably held in place, jaw surfaces 14 can be reinforced by a hard coating or a diamond sputter, or inserts made from a hard alloy, for example, an alloy based on tungsten carbide. Each of jaws 7 and 8 has a chamfered projection in the form of a tooth 15, shown in FIGS. 5 and 7 for example, that “nudges” the needle back into the lock-in zone if it ends up outside this zone for any reason, thereby preventing the needle from being dropped during operation of the suturing device.

Each clamp and/or the clamp shaft may have a range of motion such that the clamp adjusts to the arc of the needle during or after closing of the clamp so as to avoid bending of the needle with the clamp. For instance, when in the grasping position, shown for example in FIG. 7, the clamp may have a limited rotational freedom about the clamp shaft. Alternatively, the rotational freedom may be included within the shaft coupled to the clamp. The rotational freedom allows the clamp to move slightly as the clamp closes on the needle adjusting to the arc of the needle such that the clamp grasps the needle without deforming or bending the needle. An axial range of motion of the clamp and/or shaft may inhibit and/or limit distortion of the needle transverse to the arc in some embodiments. Additionally, the axial movement of the clamp and/or rotation of the clamp toward the needle may have the advantage of pushing surrounding tissue away from the needle allowing the clamp to grasp the needle and facilitating suturing of the tissue at the suture site.

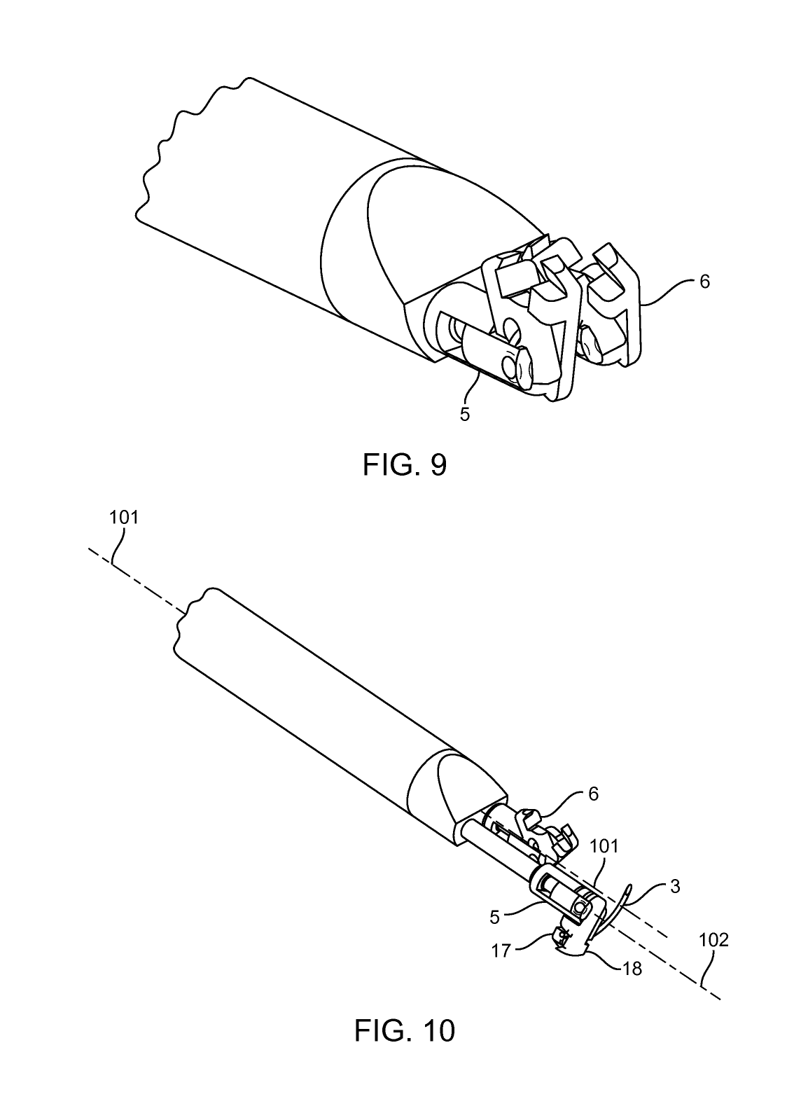

Another clamp modification is shown in FIG. 8. Here, push bar 16 has toothed projections 19, each of which is positioned in the jaw slots. Thus, as push bar 16 moves in the proximal direction, the toothed projections make jaws 17 and 18 open, as shown in FIG. 8, while moving push bar 16 in the distal direction makes the jaws close, gripping the needle in this instance. In the initial state, the clamps are in the proximal position as the device passes through a trocar, the jaws being turned as shown in FIG. 9 for example. Here, the surgeon holds the device by handle 20 (FIG. 1). When movable handle 21 is pressed, one of the clamps advances to the extreme distal position and turns around the axis 102 of the clamp's shaft. Jaws 17 and 18 then close and grip needle 3, as in FIG. 10 for example. The surgeon may then push the needle 3 through the tissue by moving or rotating the body of the device about an axis 101 of the device.

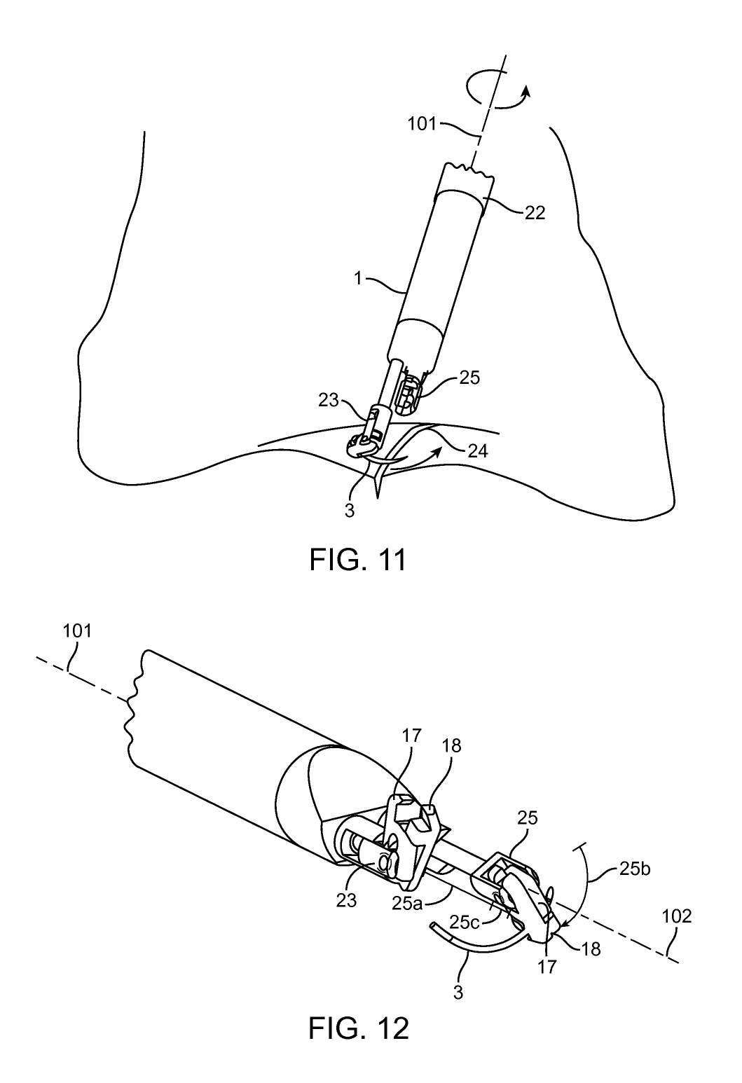

The suture application process is depicted in FIG. 11, where the working part of distal portion 1 is shown inserted through a trocar 22, while clamp 23 holds needle 3 in place. Rotating the device around the axis of the device 101, the surgeon pierces the tissue with the needle near the incision and draws needle 3 through the tissue in order to complete the suture stitch. When movable handle 21 is pressed again, the second clamp 25 advances to the extreme distal position and turns around the shaft, as shown in FIG. 12. Jaws 17 and 18 of the second clamp 25 close and grip needle 3 near its sharp end. Then, the first clamp 23 opens its jaws 17 and 18, freeing the needle, then turns around its shaft and moves into its initial state. Thus, the needle is engaged and the device now holds the needle near its sharp end, allowing the surgeon to pass the base portion of the needle and the thread 4 through the puncture. Subsequently pressing the movable handle results in clamp 23 gripping the needle at its base and the opening of clamp 25. Thus, by successively pressing movable handle 21, the surgeon engages the needle with alternating clamps and has the ability to apply a suture stitch without using additional instruments. This not only leaves the surgeon's other hand free, but also considerably expedites the suture application process.

Four exemplary and largely sequential components of the motion of the clamp between the retracted and grasping configurations are also shown schematically in FIG. 12. Specifically describing the motion from the retracted configuration, the axial movement of clamp 25 along the axis 101 of the device and the shaft supporting the clamp is schematically shown by axial line segment 25a. Once the axial movement of the jaws is sufficiently completed so that the needle is aligned within an opening between jaws 17 and 18, the jaws rotate 25b about an axis 102 of the shaft supporting clamp 25 so as to position needle 3 between jaws 17 and 18 along the axis of the shaft. As the rotational axis of the shaft is offset from the needle-engaging surfaces of the jaws, both the axial movement of the clamp and the rotational movement of the clamp help to provide clearance between the retracted clamp and the needle. The clamp 25 then again moves axially by a short distance 25c (typically less than 20 diameters of the needle, often by just a few diameters of the needle) so as to stress the needle slightly while the needle is held in the other clamp once the jaws close, thereby walking the needle back toward a desired nominal position relative to the clamp. This slight axial movement of the clamp or “overshoot” of the clamp positions the needle before the clamp closes to grasp the needle. Lastly, jaws 17 and 18 close so as to axially engage and grasp the needle, shown by the curved line segment 25d in FIG. 7. While closing on the needle, the clamp 25 may rotate slightly within a limited range of motion to adjust to the arc of the needle to prevent bending of the needle. Note that portions of these steps may overlap somewhat, so long as the associated functions performed by the motions (providing axial clearance, providing rotational clearance, stressing the needle, and grasping the clamps) are retained. Some embodiments may forego the stressing of the needle, and the order of these steps will generally be reversed when moving the clamp from a needle grasping configuration to a retracted configuration.

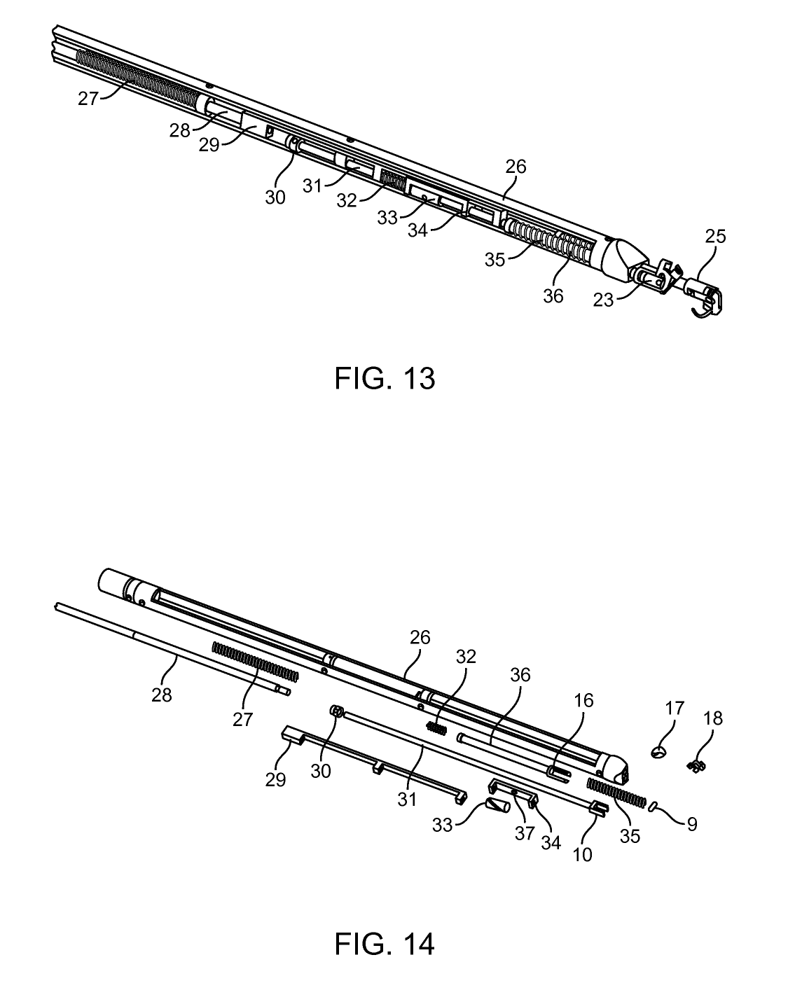

A linkage mechanism, the first part of which is housed in distal portion 1 (FIG. 1) and the other part of which is housed in proximal portion 2, is responsible for the functioning of the device. Here, the first part of this mechanism supports the clamp turning and needle gripping sequence, while its second part ensures clamp changeover and the transmission of needle gripping force from movable handle 21 to the clamps. The arrangement of the mechanism of one of the clamps located in the working part inside a frame 26 is shown in FIGS. 13 and 14 for example. Clamp jaws 17 and 18 on pin 9 are inserted into yoke 10, which is attached to a shaft 31 that is housed in a tube 36 to one end of which push bar 16 is secured. A spring 35 is located on this tube, while push bar 29 and push bar 34, inside which a helical bushing 33 is secured to shaft 31, are slipped over shaft 31. A stop plate 30 is then secured to the distal end of shaft 31. Push bar 29 has three projections with holes, a spring 32 is slipped over shaft 31 between the middle projection and push bar 34, and a rod 28 is inserted into the hole in the proximal projection of push bar 29, over which a spring 27 is slipped, one end of which rests on push bar 29, while the other end rests in the projection on rod 28. When force is applied to rod 28, it begins to move in the distal direction, compressing spring 27 and setting attached push bar 29 into motion, the middle projection of which in turn, by way of previously compressed spring 32, makes push bar 34 move in the distal direction.

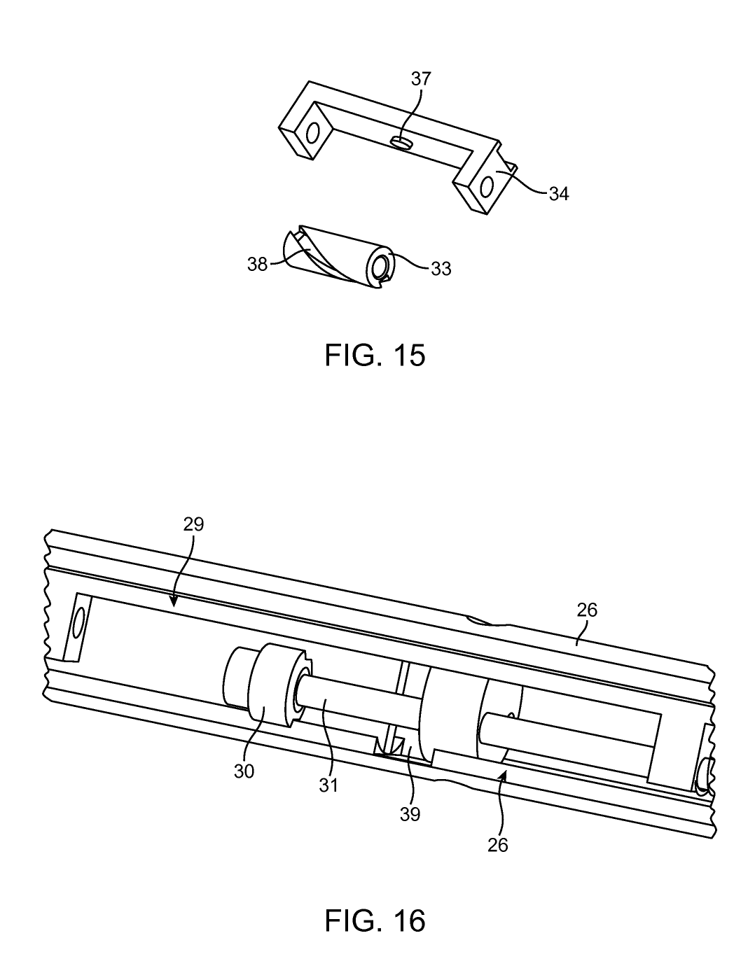

As depicted in FIGS. 13-16, push bar 34 has a projection 37 on its inner surface that moves within the helical groove of helical bushing 33 as push bar 34 is pushed in the distal direction. Bushing 33 is not able to rotate around its axis, as push bar 34 moves, since it is firmly attached to clamp shaft 31, on the proximal end of which stop plate 30 is located. Thus, the movement of push bar 29 sets shaft 31, along with jaws 17 and 18 secured in yoke 10, into motion. When stop plate 30 reaches its extreme distal position, it lines up with opening 39 in frame 26 causing subsequent movement of push bar 34 to turn bushing 33 together with shaft 31, which in turn compresses spring 21 while attached jaws 17,18 rotate around the axis 102 of shaft 31.

During subsequent movement of compression rod 28, the distal end of push bar 29 bears on tube 36, to which push bar 16 is attached, which makes the jaws close and grip the needle, and compress spring 35. Thus, over the course of a single motion of rod 28 in the distal direction, the clamp advance to a position where a proximal jaw is positioned proximal the needle along an axis of the device and the distal jaw of the clamp is positioned distal of the needle along the axis of the device. The clamp then rotates about axis 102 of the clamp shaft so that the needle 3 is between the proximal and distal jaws, which then close to grasp the needle. The reverse motion of rod 28 occurs under the influence of spring 27, whereupon push bar 29 releases tube 36, which moves in the proximal direction under the influence of spring 35, and push bar 10 opens the jaws, freeing the needle. Then the motion of push bar 29 and push bar 34 makes bushing 33, together with shaft 31 and jaws 17 and 18, return to a position where the stop plate 30 turns in opening 39 under the influence of helical bushing 33 to a position at which shaft 31, together with jaws 17 and 18, can move in the proximal direction to the initial position. Thus, the device undergoes reverse motion, during which jaws 17 and 18 of a clamp are returned to the initial position. Although the operation of one of the device's clamps, 23, has been examined here, the second clamp 25 works in a similar manner.

In the preferred embodiment, pressing the handle 21 (FIG. 2) one time causes the mechanism to move rod 28 in the distal direction and lock in the extreme position while at the time of its locking, a second rod, 40, is released and returns to the initial proximal position under the influence of a spring. When handle 21 is pressed again, rod 40 moves and locks in the extreme distal position, while rod 28 is released and returns to the initial proximal position. This functionality is realized by the linkage mechanism housed within proximal portion 2.

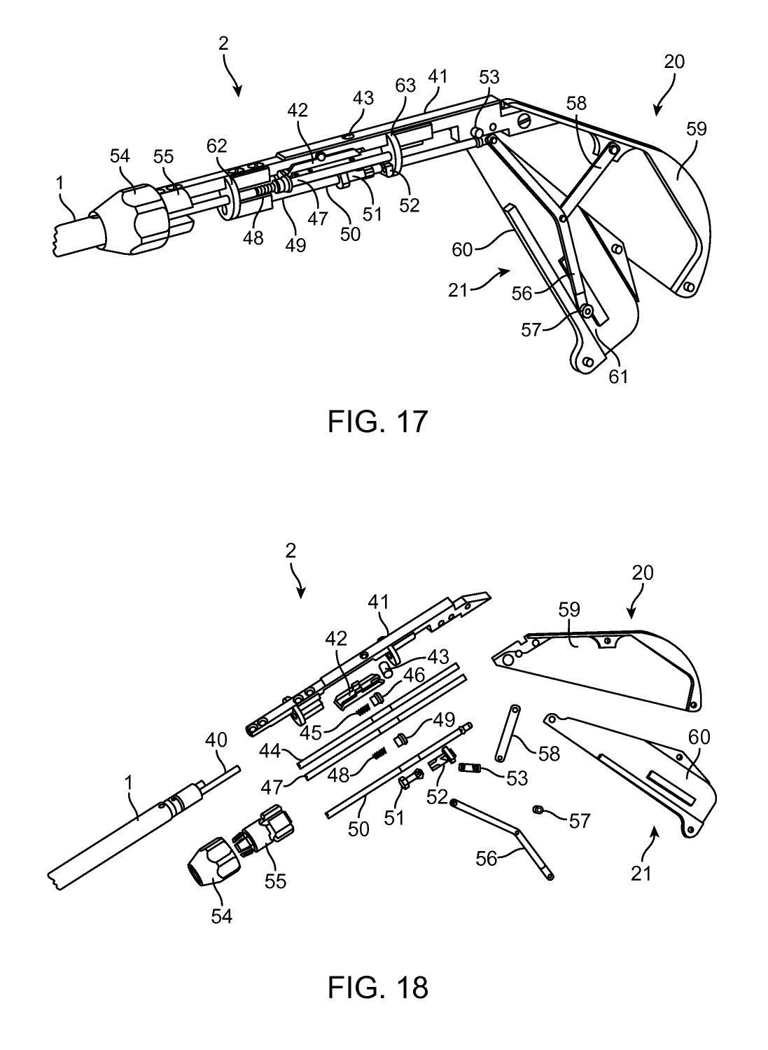

An exemplary linkage mechanism housed in proximal portion 2 is shown in FIGS. 17 and 18. Proximal portion 2 consists of a frame 41 that is rigidly connected to a grip 59, and to the shaft 53 of which a movable handle 60 is attached. Frame 41 has two plates 62 and 63 each of which has three holes. Rods 44 and 47, with springs 45 and 48 and conical bushings 46 and 49 installed thereon, are inserted into two of the holes, while a rod 50, having a rotor 51 installed thereon, is inserted into the third hole. The end of this rod is pivotally connected to a lever 56, on the other end of which a roller 57 is installed. In its midsection, lever 56 is pivotally connected to one end of a lever 58, the other end of which is pivotally connected to grip 59. An assembly for securing distal working part 1, for example, in the form of a collet 55, with a nut 54, is installed on the distal end of frame 41. A locking device 42, with a release button 43, is installed on the shaft in the midsection of frame 41.

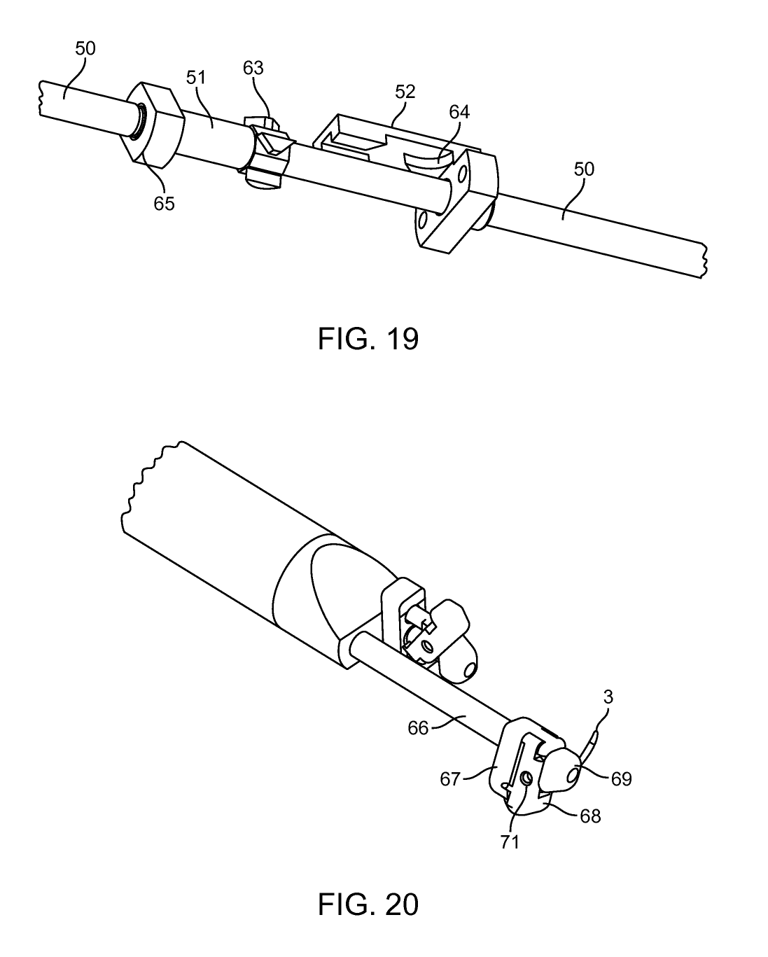

Referring now to FIG. 19, a spline 52, installed on a wall of frame 41, causes rotor 51 to rotate 90 degrees around its axis as rod 50, along with rotor 51, move past spline 52 into an extreme proximal position. An exemplary spline 52 has oblique projections 64 that cause rotor 51 to rotate around its axis. Rotor 51, installed on rod 50, has four oblique projections 63. In the initial position, handle 60 is rotated away from grip 59, and lever 56 is in a position where rod 50 is shifted into the extreme proximal position. Roller 57 of lever 56 is able to move within the confines of slotted recess 61 (FIG. 17).

The operation of the mechanism is now described with rods 44 and 47 positioned in the extreme proximal position. When handle 60 is pressed, lever 56 rotates around the shaft of its pivotal connection to lever 58. An end of lever 56 bears on rod 50, causing it to move in the distal direction, while roller 57 on the other end of lever 56 moves within slotted recess 61. Rod 50 sets rotor 51 into motion in the distal direction, whereupon the latter's projection in turn pushes one of the conical bushings. For example, bushing 49, together with rod 50, bears on rod 40 (FIG. 17) of distal working portion 1 making the appropriate clamp advance and grip the needle. When handle 60 is in the extreme depressed position, bushing 49 compresses spring 48, providing the requisite needle gripping force, and locking device 42 fixes bushing 49 in this position. Handle 60 can then be released, and returns to its initial open position. At this point, the needle is still gripped and the surgeon can use the needle to penetrate tissue next to the incision. When handle 60 is released, rod 50, together with rotor 51, moves in the proximal direction under the influence of lever 56. As rotor 51 moves in the proximal direction, the extreme proximal position rotates around its shaft 90 degrees under the influence of oblique projections 64 and 63. Thus, when handle 60 is subsequently pressed, rotor 51 sets the second conical bushing 46 into motion, which in turn makes the second clamp advance forward and grip the needle. Locking device 42 fixes the bushing in the extreme distal position and bushing 49 is simultaneously released, at which time the first clamp releases the needle and returns to the initial position under the influence of spring 27, which is located in distal portion 1. The surgeon can then extract the needle from the tissue, drawing the thread through the puncture and completing the stitch. When suturing is complete, the needle can be released and removed from the patient. This is accomplished by pressing button 43, which causes lock 42 to rotate and release both clamps, thereby releasing the needle to facilitate removal.

Distal portion 1, the working portion of the device, may be realized as a replaceable part, for instance, to be designated as a disposable component, to facilitate sterilization of the device. In this case, the replaceable part may be attached using a collet 55 with nut 54, as shown in FIG. 18. An alternative method of attaching the replaceable part is to utilize, for example, a bushing with a latch. In any case, attachment of distal portion 1 to the proximal portion 2 can be reliable and the steps for attaching and detaching it can be straightforward. Another reason to make distal portion 1 detachable is to allow for use of a set of different distal portions, which may all be attached to proximal portion 2. These differing distal working portions can differ in shape and dimensions of the jaws, length of the distal portion, and other characteristics specific to use of needles of different shapes and sizes, and suturing of a variety of tissues at different depths.

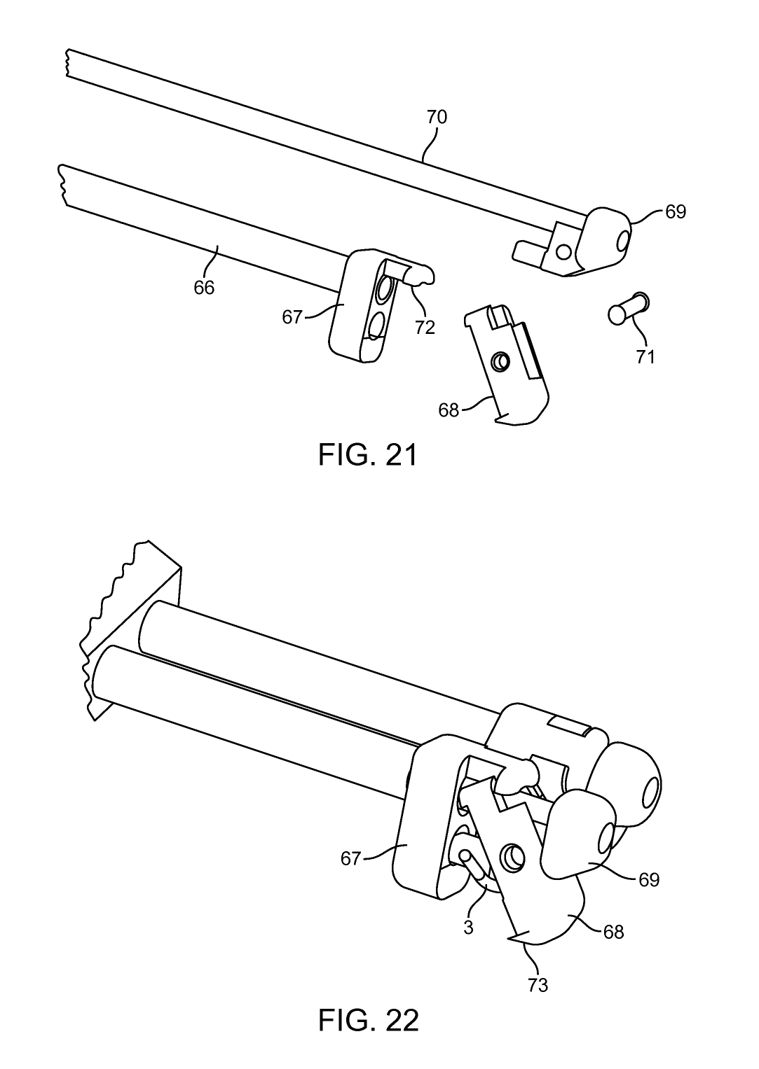

Another embodiment of the clamp is shown in FIGS. 20-22. Jaw 67 is attached to the end of tube 66, which is in turn slipped over shaft 70, to the end of which bracket 69 is attached. Movable jaw 68 is positioned on pin 71, which is inserted into aperture of bracket 69. As jaw 67 moves in proximal direction along the clamp shaft 70, its protrusion 72 bears on the upper part of jaw 68, causing it to rotate on the pin 71. This causes lower parts of jaw 67 and 68 to become more distant from each other, and the clamp opens. When jaw 67 moves in reverse (in distal direction), the clamp closes on and, in the extreme position, grips needle 3. The bottom parts of jaws 67 and 68 have flat portions, strengthened by common means (diamond sputtering, braizing of tungsten carbide plates, etc.), so the closure of the jaws results in reliable retention of needle 3 during suturing.

Reliable retention is also facilitated by the parallel alignment of the needle-gripping jaw surfaces to each other and to the needle's plane of curvature. For this reason, the device can be used with needles of varying sizes and shapes, without compromising the reliability of needle retention. In other regards, the device operates in the same manner as described above. In the initial state, a clamp is located in the extreme proximal position with the jaws opened and rotated upwards (upper clamp in FIG. 20), so as the clamp is advanced distally, the jaws clear the needle, and then rotate about the axis 102 of shaft 70 only in the extreme distal position of the clamp. Then jaw 67 continues to move distally, pressing on the needle and simultaneously causing the needle to press on jaw 68, until the jaws link and needle is gripped. This embodiment of a clamp achieves a more reliable grip on the needle because jaw 68 acts as a lever rotating about pin 71 and transfers force from jaw 67 to the needle, the force transfer ratio being significantly greater than 1:1, depending on the dimensions of the jaw 68. In the course of suturing, the needle may be subjected to a variety of factors, causing it to deform, or move in the grip of the clamp. To prevent excessive displacement of the needle, jaw 68 has a tooth-shaped protrusion 73 that nudges the needle into a position in which it can be reliably gripped.

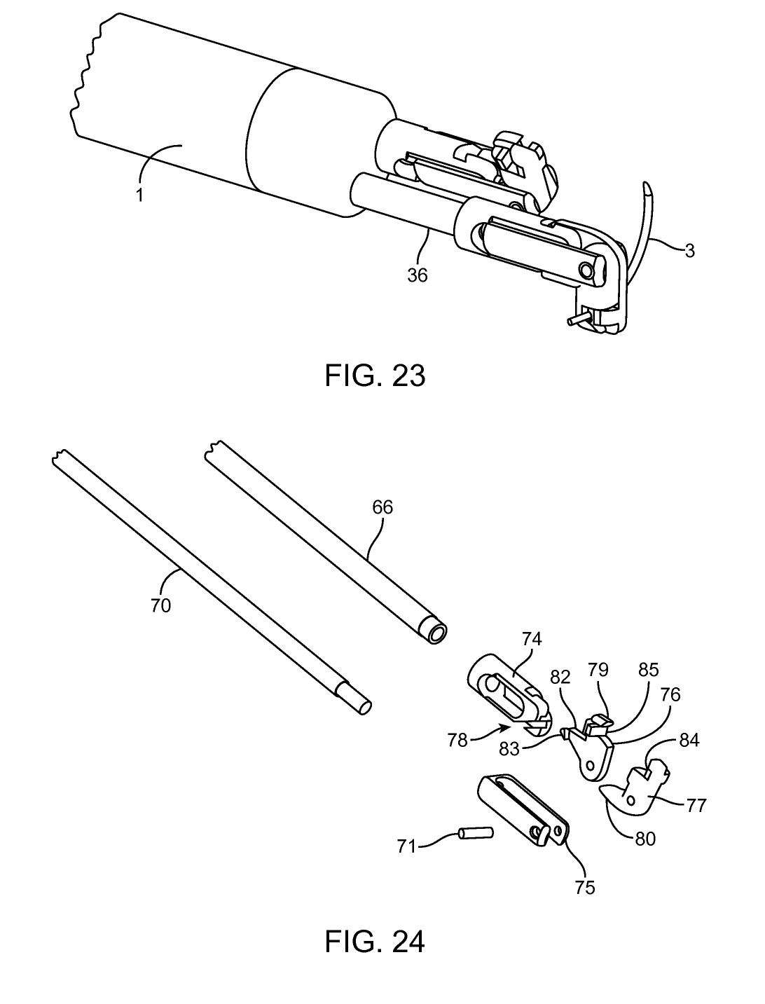

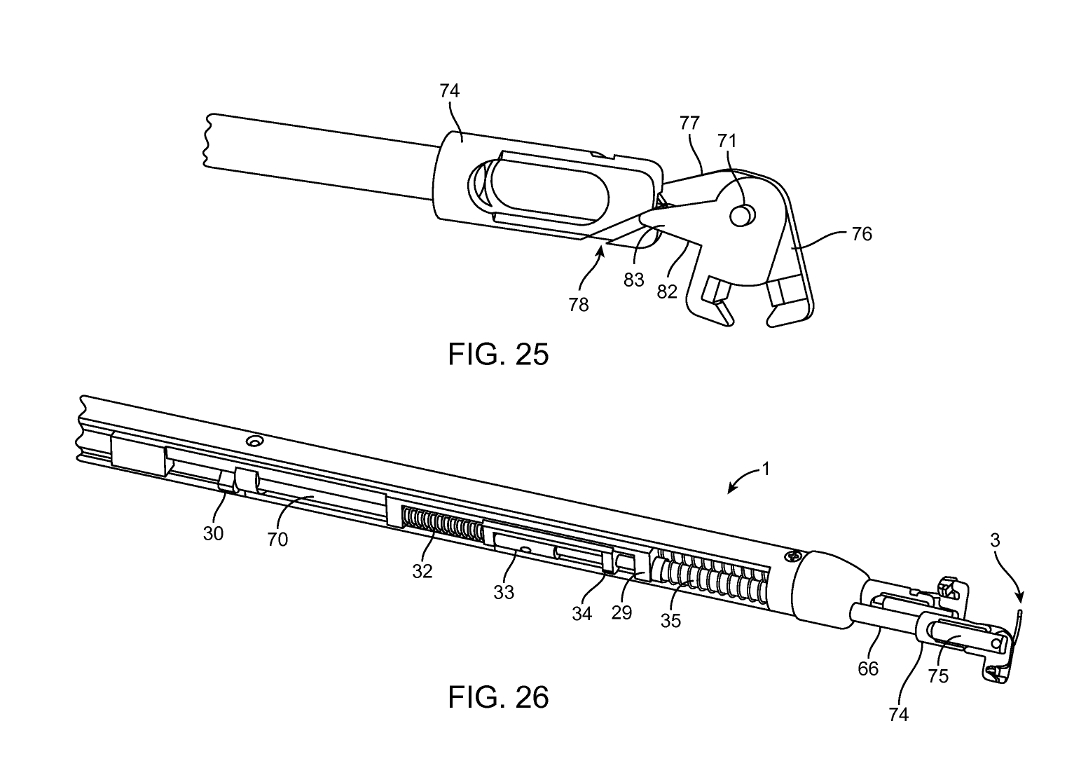

Another embodiment of a clamp is shown in FIGS. 23-24. All parts of the device, with the exception of those related to the clamp are the same, as in the previous embodiment. Unlike the previous embodiment, jaws 76 and 77 are mounted on a common pin 71 that is inserted into yoke 75, which is attached to the proximal end of shaft 70. Jaws 76 and 77 can rotate within yoke 75 about pin 71, and together with yoke 75 about the axis 102 of shaft 70, located within tube 66, and which has push bar 74 attached to its distal end. These parts are assembled in such manner that the base of yoke 75 is positioned within a slotted recess of the push bar 74, so that yoke 75 can move forward and backward along the length of the slotted recess. The slotted recesses of the push bars belonging to the two clamps are located at an angle relative to each other. Push bar 74 has two channels, angled in relation to the rod's axis. Jaw 76 has lever 82 with tip 83, and jaw 77 has lever 80 with tip 81. When assembled, the tips of the levers are placed into the angled channels 78 of push bar 74, as illustrated in FIG. 25 with the yoke removed.

In many embodiments, surfaces 84 and 85 of jaws 76 and 77 are strengthened to help the jaws maintain a reliable grip on the needle. Surfaces 84 and 85 may be strengthened with inserts made of hard material, such as tungsten carbide, or diamond sputtering. In certain embodiments, the jaws also have claw 79 that corrects the needle's position if it is deviated from the normal in process of gripping. As push bar 74 moves distally, tips 81 and 83 travel along the inner surface of the angled channels of push bar 74. This causes jaws to rotate about the pin 71 and the working surfaces 84 and 85 of jaws 76 and 77 to come together. As push bar 74 moves proximally, tips 81 and 83 travel along the outer surface of the angled channels 78 of push bar 74 causing the jaws to open. The described design of jaws in conjunction with push bar with angled channels allows a significantly greater force to be transmitted to the jaws, and thus ensures a more reliable grip on the needle. This is achieved by converting the longitudinal force applied by the handle to the push bar into torque at the jaw levers. In effect, as the angle is decreased, the angled channels form a wedge, which has a higher transmission factor.

In other regards, the clamps of the embodiment of FIGS. 23-26 operate in the same manner as described in previous embodiments. In the initial state, both clamps are rotated upward, and are located proximally. As the handle is pressed, one of the clamps advances distally to its extreme distal position. As it moves, yoke 75 lags the push bar 74, so that jaws 76 and 77 are open. Once push bar 74, together with stop plate 30, reach their extreme distal positions, further pressure on the handle causes helical bushing 33 and shaft 70 to rotate. This rotational movement results in the open jaws being rotated about axis of push bar 70 and around a portion of the needle 3. As further pressure is applied to the handle, the distal end of the push bar 29 presses on tube 66 and push bar 74, which in turn causes the jaws to close and grip the needle. In this manner, pressing the handle once causes the jaws to advance to the needle location, rotate into position, and grip the needle. When a clamp is released, the process is reversed due to action of a compressed spring 27 (FIG. 13). Here push rod 29 releases tube 66, which under force from spring 35, and moves proximally causing push rod 74 to open the jaws and release the needle. Then, motion of push rod 29 and push rod 74 causes bushing 33, along with shaft 70 and jaws 76 and 77, to rotate to a position where stop plate 30 moves, allowing shaft 70 to move proximally to the initial proximal position.

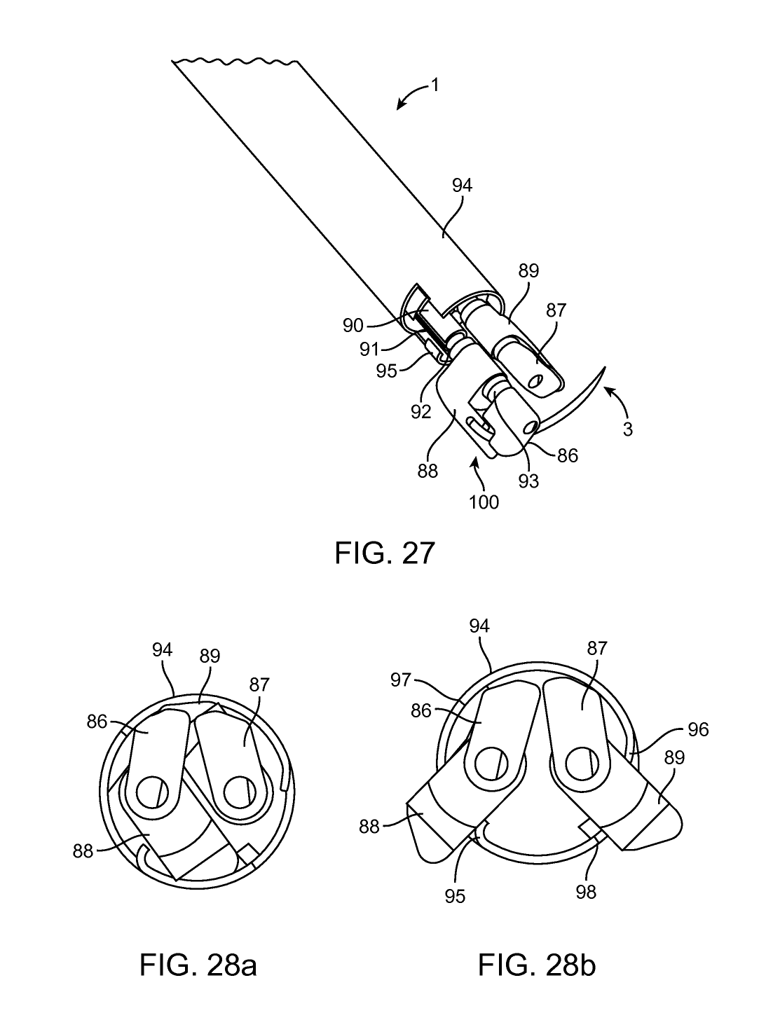

Another embodiment of the clamp is shown in FIG. 27-29. Each of two clamps consists of a rotating jaw and a power jaw, shown in FIG. 27. First clamp contains rotating jaw 86, attached to rod 92, and power jaw 88, attached to tube 90. In the same fashion, second clamp contains rotating jaw 87 and power jaw 89. Tube 90 has a longitudinal groove 91 and an annular groove 92. This type of a clamp allows use of larger needles and suturing of thicker tissue due to the ability of the power jaws to fold and unfold between a folded configuration and an unfolded working configuration. The folded configuration has a reduced profile to facilitate introduction of the clamps along with distal working portion 1 through a trocar. FIG. 28a shows an end view of the working part with the jaws in the folded configuration. In the working configuration, the jaws unfold and function as described above, alternating holding the needle. FIG. 28b shows an end view of the working part with the jaws in the unfolded configuration. A circular sheath 94, shown in FIGS. 27-28b, may be employed in order to control position of the power jaws. The sheath has tabs 95 and 96 at its tip, which can be used to fold power jaws 87 and 88 by rotating the sheath clockwise, and unfold power jaws 87 and 88 by rotating the sheath counterclockwise.

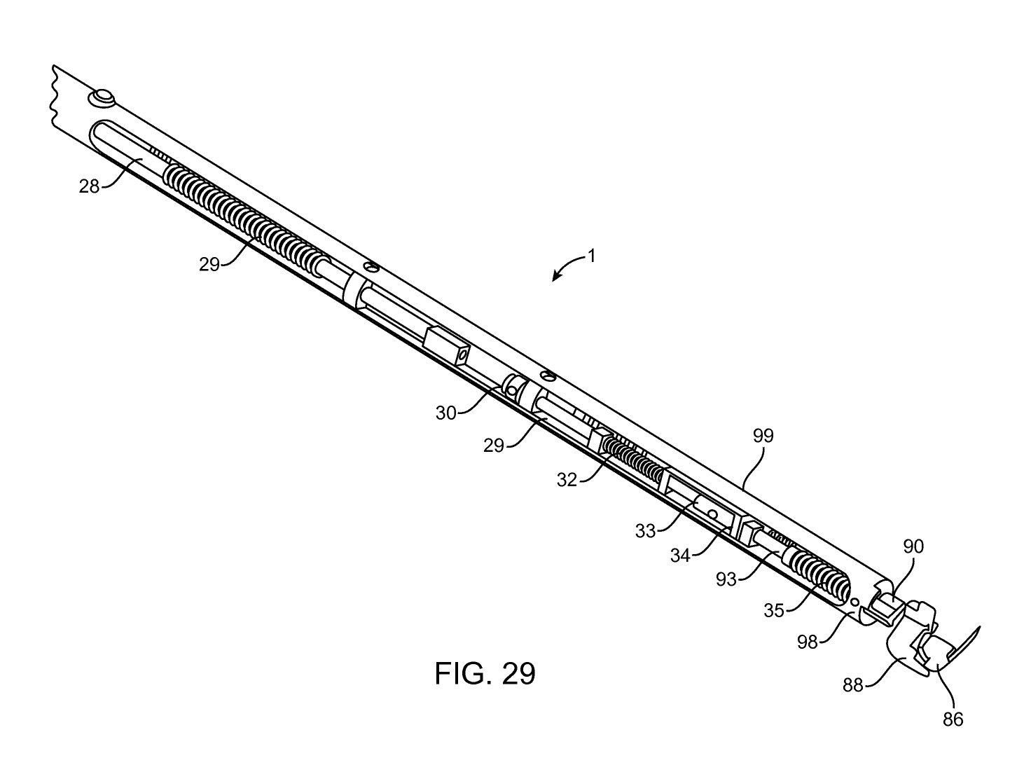

FIG. 29 shows the working part distal portion 1 with the sheath removed and frame 99, the distal portion of which has two pins 98 for the first and second clamp. The inside ends of the pins 98 protrude into the longitudinal grooves 91 of tubes 90, to prevent axial rotation of the tubes once they have been extended. On the whole, operation of the device is analogous to that described in the embodiments above, with some important distinctions. Prior to starting the procedure, surgeon rotates the sheath 94, to fold power jaws 88 and 89 into the folded configuration. At this point, rotating and power jaws are in their initial positions, with tubes 90 of both clamps forced into their extreme proximal position by force of springs 35. In this position, pins 98 are located in the annular grooves 92 (FIG. 27), so that tubes 90 with attached power jaws 88 and 89 are able to rotate about their longitudinal axes when pushed by tabs 95 and 96 (FIG. 28a, 28b). After the working part has been introduced through trocar, the surgeon rotates sheath 94, and tabs 95 and 96 cause power jaws 88 and 89 to unfold into the working configuration. In order to grip the needle, the surgeon presses movable handle 21 (FIG. 1), and the force is transferred to rod 28, causing the push rod 29, and push rod 34 through spring 32, to move distally. This motion is passed to bushing 33, attached to rod 93, so rotating jaw 86, attached to the distal end of rod 93, is advanced to its extreme distal position. At this point, stop plate 30 reaches its extreme distal position, where helical bushing 33 rotates rod 93 and rotational jaw 86 about the longitudinal axis, and the jaw reaches its working position near the needle 3. As rod 28 continues its distal motion, the distal end of push bar 29 bears on the end of tube 90 causing it and the power jaw 88 attached to its distal end to move distally, pressing needle 3 against the rotating jaw 86. Power jaw 88 has a diagonal lug 100 (FIG. 27), which serves to correct the needle position if it deviates from its normal position. In the extreme distal position of the power jaw 88, bushing 49 of the handle is fixated by lock 42 (FIG. 17) and the needle is firmly gripped between jaws 86 and 88. In this position, the surgeon can penetrate tissues with the needle. When moving handle 21 is pressed again, the needle is gripped by the second clamp, and almost simultaneously released by the first clamp, and the jaws of the first clamp return to the initial proximal position. At this point, the needle is gripped near its tip by the second clamp and the surgeon can complete the stitch and pull the suture through the puncture.

After completing the procedure, the surgeon rotates sheath 94 to fold power jaws into the folded configuration, so that the working part can be extracted through the trocar. The described embodiment of the power clamps differs from those above in that the jaws are made to be more powerful, to grip the needle more securely, and in that the unfolding design of the power jaws allows gripping the needle with greater distance between the clamps, allowing deeper penetration of tissue in suturing.

What is claimed is:

1. A suturing method comprising:
introducing a body of a suturing device to a surgical site for a patient, the body extending along a device axis toward first and second clamps;
grasping a base portion of a needle between a distal jaw element and a proximal jaw element of the first clamp so that the needle is disposed along the device axis between the proximal jaw element and the distal jaw element, the needle having a needle axis extending from the base portion to a tissue penetrating portion having a sharp end;
inserting the sharp end of the needle through a tissue of the patient while the first clamp holds the needle at a fixed location relative to the body;
advancing the second clamp toward the needle by translating a shaft supporting the second clamp along the device axis and rotating the second clamp about the shaft;
grasping the tissue penetrating portion of the needle with the second clamp of the suturing device;
releasing the base portion of the needle from the first clamp and withdrawing the first clamp away from the needle by translating a shaft supporting the first clamp along the device axis and rotating the first clamp about the shaft;
pulling the base end of the needle through the tissue by moving the body of the suturing device while the second clamp holds the needle.

2. The suturing method of claim 1, wherein inserting the sharp end of the needle through the tissue and pulling the base end of the needle through the tissue each comprise moving the body of the suturing device by a physician manually holding a proximal portion of the suturing device.

3. The suturing method of claim 1, wherein grasping a portion of the needle comprises:
advancing the first or second clamp axially along the device axis toward the needle to a position lateral of the needle;
rotating the clamp laterally toward the portion of the needle once the needle is disposed axially between the jaw elements of the clamp such that the clamp partially surrounds the portion of the needle; and
closing the clamp on the portion of the needle.

4. The suturing method of claim 3, further comprising axially stressing the needle along the axis of the needle with the clamp before closing the clamp on the portion of the needle.

5. The suturing method of claim 1, wherein grasping a portion of the needle comprises:
advancing the first or second clamp axially along the device axis toward the needle to a position lateral of the needle;
rotating the clamp laterally toward the portion of the needle such that the
clamp partially surrounds the portion of the needle such that the proximal jaw element is proximal of the portion of the needle and the distal jaw element is distal of the portion of the needle;
advancing the clamp axially along the device axis slightly to contact the needle with the proximal portion of the needle to axially stress the needle; and
closing the clamp on the portion of the needle.

6. The suturing method of claim 1, wherein the steps are performed in the order in which the steps are written in the claim.

7. The suturing method of claim 1, wherein grasping the portion of the needle with the first or second clamp further comprises applying a gripping force on opposing outer surfaces of the portion of the needle by closing proximal and distal jaw elements of the clamp on the needle.

8. The suturing method of claim 7, wherein grasping a portion of the needle further comprises applying a gripping force on the portion of the needle with the clamp substantially in the direction of the device axis.

9. The suturing method of claim 1, further comprising simultaneously holding the portion of the needle near the base end and the portion of the needle near the sharp end with the first and second clamps before releasing the portion of the needle with the first clamp.

10. The suturing method of claim 9, wherein simultaneously holding the needle further comprises increasing a gripping force of the second clamp while simultaneously decreasing the gripping force of the first clamp.

11. The suturing method of claim 10, wherein the increase in the gripping force of one clamp roughly corresponds to the decrease in the gripping force of the other clamp.

12. The suturing method of claim 1, wherein inserting the distal portion of the device into the body cavity further comprises expanding the clamps from a folded configuration into a working configuration after the clamps pass through the trocar.

13. The suturing method of claim 12, wherein expanding the clamps from a folded configuration into a working configuration comprises moving a sheath relative to the body, the sheath constraining the clamps when in the folded configuration so as to facilitate insertion through the trocar and being moved to allow unfolding of the clamps to the working configuration.

14. The suturing method of claim 13, wherein expanding the clamps from a folded configuration into a working configuration comprises distally advancing the clamps beyond a sheath, the sheath having constrained the clamps when in the folded configuration so as to facilitate insertion through the trocar.

15. The suturing method of claim 12, further comprising:
releasing a locking mechanism that holds the clamps in the working configuration; and
folding the clamps from the working configuration to a folded configuration to facilitate removal of the trocar.

16. The suturing method of claim 1, wherein the grasping and releasing steps are effected by actuating a handle of the suturing device with a hand of a surgeon.

17. The suturing method of claim 1, wherein grasping and releasing of the clamps are effected by moving a handle between a first configuration relative to the body and a second configuration relative to the body, movement of the handle from the first configuration to the second configuration and back to the first configuration defining a handle actuation cycle, and wherein the needle alternates at least once between being supported by the first clamp and being supported by the second clamp with each handle actuation cycle.

18. An endoscopic suturing device for use with a suturing needle, the device comprising:
a body having a proximal portion disposed and a distal portion with a device axis extending therebetween;
a first shaft extendable from the distal portion of the body, the first shaft movable axially along the device axis and rotatable relative to the body;
a first clamp supported by the first shaft, the first clamp having a proximal jaw and a distal jaw;
a second shaft extendable from the distal portion of the body, the second shaft movable axially along the device axis and rotatable relative to the body;
a second clamp supported by the second shaft, the second clamp having a proximal jaw and a distal jaw; and
a linkage effecting movement of each of the first and second clamps between a retracted position and a grasping position by axial and rotation movement of the shafts, each clamp grasping a portion of the needle in the grasping position so that the needle is disposed along the device axis between the proximal jaw and the distal jaw, and each clamp is rotated laterally away from the needle and retracted proximally away from the needle in the retracted position to increase clearance between the retracted clamp and the tissue and/or needle.

19. The device of claim 18, each of the first and second shafts having a shaft axis extending distally from the distal portion of the body, and wherein the linkage, when moving a clamp from a retracted position to a grasping position, effects movement of the clamp by:
axial advancement of the first or second clamp along the device axis;
rotation movement of the axially advanced clamp about the shaft axis so that the needle is disposed between the proximal jaw and the distal jaw along the device axis; and
closing of the clamp on the needle so that the needle is held between the proximal jaw and distal jaw of the clamp.

20. The device of claim 19, wherein the linkage effects a slight axial movement of the clamp so that the proximal jaw engages the needle before closing the clamp on the needle and after rotation movement of the clamp about the shaft axis.

21. The device of claim 19, wherein the linkage, when moving a clamp from a grasping position to a retracted position, effects movement of the clamp by:
opening of the first or second clamp so as to release the needle between the proximal jaw and distal jaw of the clamp along the device axis;
rotation movement of the clamp about the shaft axis so as to move the clamp away from the needle; and
axial movement of the clamp away from the needle along the device axis.

22. The endoscopic suturing device of claim 18, wherein each clamp, proximal and distal jaw and shaft is rigid.

23. The endoscopic suturing device of claim 18, wherein the first and second clamp, the first and second shaft and the linkage effecting movement of each of the first and second clamps comprise a detachable cartridge to facilitate sterilization or customization of the device with different cartridges.

24. The endoscopic suturing device of claim 22, wherein the linkage effecting movement of each of the first and second clamps comprises a compression rod.

25. The endoscopic suturing device of claim 25, wherein the linkage effecting movement further comprises springs.

26. The endoscopic suturing device of claim 18, further comprising a handle disposed at the proximal end of the body and coupled to the first and second clamps by the linkage so that an actuation cycle of the handle alternates between:
the first clamp in the grasping position and the second clamp in the retracted position; and
the second clamp in the grasping position and the first clamp in the retracted position.

27. The endoscopic suturing device of claim 26, wherein each clamp opens after the other clamp has closed during the handle actuation cycle so that the clamps maintain the needle at a substantially fixed location relative to the body.

28. The endoscopic suturing device of claim 26, wherein each clamp is closed on the needle during a portion of the handle actuation cycle.

29. The endoscopic suturing device of claim 26, wherein one clamp exerts an increasing force on the needle while the other clamp exerts a decreasing force on the needle during the portion of the actuation cycle when both clamps are closed on the needle.

30. The endoscopic suturing device of claim 18, wherein the linkage effects advancement of the first or second clamp toward the needle and rotation of the clamp laterally toward the needle.

31. The endoscopic suturing device of claim 18, wherein each of the proximal and distal jaw elements of the first or second clamp comprise a gripping surface, the gripping surfaces positioned to grip almost entirely in the axial direction.

32. The endoscopic suturing device of claim 31, further comprising a chamfered projection disposed at an end of a jaw element to nudge a needle into alignment with the gripping surfaces of the jaws during grasping of the needle.

33. The endoscopic suturing device of claim 18, wherein the first or second clamp assembly has a folded configuration and a working configuration, the clamp having a reduced profile to facilitate introduction through a trocar into a body cavity of a patient in the folded configuration and the clamp unfolding to facilitate operation of the suturing device in the working configuration.

34. The endoscopic suturing device of claim 33, further comprising a sheath, wherein the sheath houses the clamps in the folded configuration during insertion through the trocar and wherein the sheath is positioned proximally of the clamps when the clamps are in the working configuration.

35. The endoscopic suturing device of claim 18, wherein the distal portion of the suturing device is removably coupled to the suturing device.
